(19) United States
(12) Patent Application Publication
(10) Pub. No.: US 2024/0209282 A1
(43) Pub. Date: Jun. 27, 2024

(54) N-ACYL AMINOALKANE SULFONATE SURFACTANTS AND DERIVATIVES THEREOF

(71) Applicant: The Procter & Gamble Company, Cincinnati, OH (US)

(72) Inventors: Victor Manuel ARREDONDO, West Chester, OH (US); Karunakaran NARASIMHAN, Mason, OH (US)

(21) Appl. No.: 18/530,397

(22) Filed: Dec. 6, 2023

Related U.S. Application Data
(60) Provisional application No. 63/430,692, filed on Dec. 7, 2022.

(51) Int. Cl. C11D 1/12; C11D 3/20

(52) U.S. Cl. CPC C11D 1/12; C11D 3/201; C11D 3/2093

(57) ABSTRACT

A surfactant composition includes greater than 75 wt. % N-acyl aminoalkane sulfonate of formula (I). The surfactant composition is substantially free of solvent and NaCl. A process for preparation of a mixture comprising a N-acyl aminoalkane sulfonate surfactant includes combining (a) an aminoalkane sulfonic acid of formula (II) or (b) an anhydrous alkali salt of an aminoalkane sulfonic acid of formula (II), a waterless base, and a fatty alkyl ester of formula (III) to form the mixture including N-acyl aminoalkane sulfonate of formula (I). The process further includes increasing the temperature of the mixture to 190° C. or less, preferably 170° C. or less, more preferably 160° C. or less to form a reaction mixture; and continuously removing alkyl alcohol from the reaction mixture.

### TECHNICAL FIELD

The present disclosure relates generally to N-acyl aminoalkane sulfonate surfactants and derivatives and, in particular embodiments, N-acyl aminoalkane sulfonate surfactant compositions with low amounts of impurities.

### BACKGROUND

Surfactants are the single most important cleaning ingredient in cleaning products. Environmental regulations, consumer habits, and consumer practices have forced new developments in the surfactant industry to produce lower cost, higher-performing, and environmentally friendly products.

Surfactants are key ingredients playing important roles in a variety of applications and consumer products such as in detergents, hard surface cleaners, fabric softeners, body wash, face wash, shampoo conditioners, conditioning shampoos, and other surfactant-based compositions. Many catalogs and patents describe surfactant options that can be too expensive to use. The high cost is many times due to the starting materials used to make such surfactants, inefficient reaction schemes and/or complex processes required for their manufacture to meet specific quality attributes. Accordingly, new methods are needed to produce surfactant compositions at low cost containing minimal impurities or additives.

N-acyl taurates, or N-acyl taurides as named by others, (and other amino acid-based) surfactants can be commercially manufactured from the corresponding fatty acid chlorides and amino acids using Schotten Baumann chemistry as shown in equation 1.

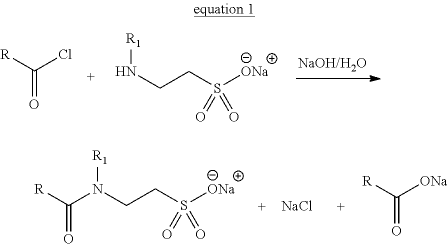

This amidation reaction is typically carried out in water, but the use of mixed water-solvent systems has been reported. Typically, the sodium N-acyl aminoalkane sulfonate surfactant formed is obtained in the form of an aqueous composition containing 20-30% active with invariably high levels of undesirable inorganic salt (NaCl). The latter can be removed via additional post-reaction steps that can add significant cost and process complexity. This surfactant-making method is expensive and requires the manufacture of fatty acid chlorides which uses chlorinating agents such as phosphorous trichloride, (PCl₃), phosphorous pentachloride (PCl₅), thionyl chloride (SOCl₂), oxalyl chloride (COCl)₂ or phosgene (poisonous gas). These chlorinating agents are quite reactive, can be toxic, might require very special handling and metallurgy. Furthermore, depending on the specific chemistry and process used, separating the fatty acid chlorides away from byproducts and catalysts used has been difficult to solve. Thus, the products may contain undesired impurities that can be carried through to the synthesis of the corresponding surfactant.

The preparation of N-acyl taurates has also been reported to occur by the direct condensation of carboxylic acid with 2-aminoalkane sulfonic alkali salts as shown in equation 2. For this reaction to take place, however, the removal of water and the use of high temperatures (190-240° C.) and an inert atmosphere is necessary. This direct amidation reaction can be carried out in the presence of a catalyst such as zinc oxide, hypophosphorous acid, boric acid and others, which remain in the surfactant mixture. Decomposition byproducts have been reported resulting in poor product yields, and unacceptable product discoloration and odor. Typically, the carboxylic acid is said to be used in ≥30 molar excess relative to the taurine. To produce an N-acyl taurate which is free from fatty acid through this chemical approach, the crude reaction mixture is subjected to additional purification processing steps such as distillation, extraction, recrystallization, or combinations thereof.

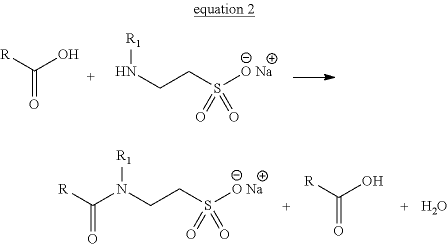

Fatty alkyl esters have also been used as starting materials. In accordance with another process a fatty alkyl ester is reacted with taurine in the presence of polyol solvent such as glycerine or propylene glycol. The relative mole ratio of polyol to the amino compound ranged from about 8:1 to about 1:1. In the lead example included, the resultant product contained 34% glycerol which remained in the surfactant mixture, which is undesired for many applications.

In summary, N-acyl aminoalkane sulfonate surfactants made using these processes tend to contain high levels of undesirable by-products, such as salt (NaCl), or solvents such as methanol, glycerol and propylene glycol. Thus, there is a need for N-acyl aminoalkane sulfonate surfactant compositions that are made under atmospheric conditions, are produced with low proportion of by-products and low levels of solvents or additives.

### SUMMARY

The present disclosure attempts to solve one more of the needs by providing a surfactant composition including greater than 75 wt. % N-acyl aminoalkane sulfonate of formula (I), by weight of the surfactant composition:

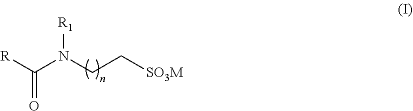

where R is an C₅-C₂₁ alkyl substituent, R₁ represents H, or C₁ to C₄ alkyl radical, n is an integer from 1 to 2, and M is a cationic group selected from the group consisting of alkali metal salts and hydrogen, and the surfactant composition is substantially free of solvent and NaCl. The present disclosure also relates to surfactant compositions that are a solid or an aqueous liquid composition.

The present disclosure further relates to a process for preparation of a mixture including a N-acyl aminoalkane sulfonate surfactant including combining: (a) an aminoalkane sulfonic acid of formula (II) or (b) an anhydrous alkali salt of an aminoalkane sulfonic acid of formula (II):

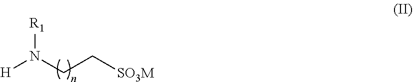

where R₁ represents H, or C₁ to C₄ alkyl radical, n is an integer from 1 to 2, and M is a cationic group selected from the group consisting of alkali metal salts and hydrogen, a waterless base, and a fatty alkyl ester of formula (III)

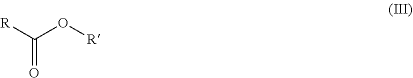

where R is selected from an C₅-C₂₁ alkyl substituent and R′ is a C₁ or higher alkyl substituent, preferably methyl, to form the mixture including N-acyl aminoalkane sulfonate of formula (I):

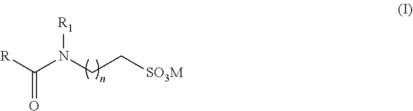

where R is an C₅-C₂₁ alkyl substituent, R₁ represents H, or C₁ to C₄ alkyl radical, n is an integer from 1 to 2, and M is a cationic group selected from the group consisting of alkali metal salts and hydrogen; increasing the temperature of the mixture to 190° C. or less, preferably 170° C. or less, more preferably 160° C. or less to form a reaction mixture; and continuously removing alkyl alcohol from the reaction mixture.

In another aspect, the disclosure is directed to a consumer product cleaning or personal care composition comprising about 0.001 wt. % to about 99.999 wt. % or about 0.1 wt % to about 80 wt. % of N-acyl aminoalkane sulfonate surfactant, as described herein, based on the total weight of the composition, and 0.001 wt. % to about 99.999 wt. % of one or more additional cleaning components, or one or more additional personal care components.

### DETAILED DESCRIPTION

Features and benefits of the present disclosure will become apparent from the following description, which includes examples intended to give a broad representation of the disclosure. Various modifications will be apparent to those skilled in the art from this description and from practice of the disclosure. The scope is not intended to be limited to the particular forms disclosed and this disclosure covers all modifications, equivalents, and alternatives falling within the spirit and scope of this disclosure as defined by the claims.

As used herein, the articles including “the,” “a” and “an” when used in a claim or in the specification, are understood to mean one or more of what is claimed or described.

As used herein, the terms “include,” “includes” and “including” are meant to be non-limiting.

The term “substantially free of” or “substantially free from” as used herein refers to either the complete absence of an ingredient or a minimal amount thereof merely as impurity or unintended byproduct of another ingredient. A composition that is “substantially free” of/from a component means that the composition comprises less than about 0.5%, 0.25%, 0.1%, 0.05%, or 0.01%, or even 0%, by weight of the composition, of the component.

As used herein, the term “solid” includes granular, powder, flakes, noodles, needles, extrudates, ribbons, beads and pellets product forms and comprise less than about 0.5%, 0.25%, 0.1%, 0.05%, or 0.01%, or even 0%, by weight of the composition, of the water.

As used herein, “personal cleansing composition” includes personal cleansing products such as shampoos, conditioners, conditioning shampoos, shower gels, liquid hand cleansers, facial cleansers, and other surfactant-based liquid compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In this description, all concentrations are on a weight basis of the composition, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

### N-Acyl Aminoalkane Sulfonate Surfactant

The N-acyl aminoalkane sulfonate surfactants disclosed herein have the following general formula (I):

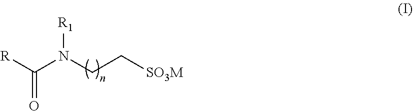

where R is an C₅-C₂₁ alkyl substituent, R₁ represents H, or C₁ to C₄ alkyl radical, n is an integer from 1 to 2, and M is a cationic group selected from the group consisting of alkali metal salts and hydrogen. Preferably, R is a C₇₋₁₇ alkyl substituent. The alkyl substituent may be branched or unbranched.

The N-acyl aminoalkane sulfonate surfactants described herein are typically not single compounds as suggested by their general formula (I), but rather, as one skilled in the art would readily appreciate, they comprise a mixture of several homologs having varied chain lengths and molecular weight. The N-acyl aminoalkane sulfonate surfactants described herein may be either saturated or unsaturated.

The N-acyl aminoalkane sulfonate surfactant composition of the present disclosure includes at least 50 wt. % N-acyl aminoalkane sulfonate surfactant by weight of the surfactant composition. For example, the composition may include from 65 to 95 wt. %, from 70 to 95 wt. %, from 75 to 95 wt. %, from 80 to 95 wt. %, from 85 to 95 wt. %, from 90 to 95 wt. %, from 65 to 90 wt. %, from 70 to 90 wt. %, from 75 to 90 wt. %, from 80 to 90 wt. %, from 85 to 90 wt. %, from 65 to 85 wt. %, from 70 to 85 wt. %, from 75 to 85 wt. %, from 80 to 85 wt. %, from 65 to 80 wt. %, from 70 to 80 wt. %, from 75 to 80 wt. %, from 65 to 75 wt. %, from 70 to 75 wt. %, or from 6d5 to 70 wt. % N-acyl aminoalkane sulfonate surfactant by weight of the surfactant composition.

The surfactant composition may include at least 5 wt. %, preferably from about 5 to about 15 wt. %, more preferably about 8 to about 10 wt. % N-acyl-N-methylaminoalkane sulfonate surfactant by weight of the surfactant composition. The surfactant composition may include from 5 to 50 wt. %, from 5 to 35 wt. %, from 5 to 25 wt. %, from 5 to 20 wt. %, from 5 to 15 wt. %, from to 12 wt. %, from 5 to 10 wt. %, from 5 to 8 wt. %, from 8 to 50 wt. %, from 8 to 35 wt. %, from 8 to 25 wt. %, from 8 to 20 wt. %, from 8 to 15 wt. %, from 8 to 12 wt. %, from 8 to 10 wt. %, from 10 to 50 wt. %, from 10 to 35 wt. %, from 10 to 25 wt. %, from 10 to 20 wt. %, from 10 to 15 wt. %, from 10 to 12 wt. %, from 12 to 50 wt. %, from 12 to 35 wt. %, from 12 to 25 wt. %, from 12 to 20 wt. %, from 12 to 15 wt. %, from 15 to 50 wt. %, from 15 to 35 wt. %, from 15 to 25 wt. %, from 15 to 20 wt. %, from 20 to 50 wt. %, from 20 to 35 wt. %, or from 25 to 50 wt. % N-acyl-N-methylaminoalkane sulfonate surfactant by weight of the surfactant composition.

The N-acyl aminoalkane sulfonate surfactant composition of the present disclosure further comprises fatty acid. The fatty acid may be present as free fatty acid or in the form of fatty acid soap. The amount in the composition may range from 1 to about 10% by weight, from 2 to 7% by weight, or from 3-5% by weight, specifically reciting all values within these ranges and any ranges created thereby.

Beneficially, the N-acyl aminoalkane sulfonate surfactant composition of the present disclosure may be substantially free of impurities including water, salt (NaCl), polyol solvents, and methanol. The composition of the disclosure may comprise less than 5%, 2%, 1%, 0.1%, substantially free, and in some instances, free of one or any combination of these impurities.

The present disclosure further encompasses concentrated compositions, often referred to as pastes, and also solids, such as powders and tablets. These concentrated compositions may be combined with various adjunct ingredients (for example, water) to make a variety of detergent products, including personal cleansing compositions and laundry detergents.

Typically, inorganic salt (NaCl) is added to cleansing formulations made with sulfated surfactants to thicken the product. It has been surprisingly found that adding inorganic salt to the formulas that are substantially free of sulfated surfactants and/or using sulfate-free surfactants containing high inorganic salt in the presence of cationic conditioning polymer can cause product instability due to formation of a gel-like surfactant-polymer complex in the composition. Thus, it is desirable to avoid or minimize adding NaCl to the formula and/or use low inorganic salt (NaCl) containing raw materials. Commercially available sulfate-free surfactants such as sodium methyl cocoyl taurate (cocoyl aminoalkane sulfonate), and other amino acid-based surfactants, typically come with high levels of inorganic salt such as 5% or higher. Use of these high salt (such as, NaCl) containing raw materials in sulfate-free surfactant-based cleaning formulations can cause formation of undesired gel-like surfactant-polymer complex in the product before use. The surfactant composition described herein may enable the formulation of stable cleansing products substantially free of sulfated surfactants.

### Process of Making N-Acyl Aminoalkane Sulfonate Surfactants

The process described herein allows for the preparation of N-acyl aminoalkane sulfonate surfactants having low levels of impurities. The conventional Schotten-Baumann acid chloride route to N-acyl aminoalkane sulfonate surfactants—generates NaCl and other impurities, thereby yielding an undesirable output. Further, other reactions for making N-acyl aminoalkane sulfonate surfactants use a low boiling point solvent and are carried out in closed reactors under pressure, and not under atmospheric conditions. High pressure reaction conditions are inherently more dangerous, time consuming, complicated and costly and are, therefore, not desirable. Others have used high boiling solvents such as polyols, glycerol and propylene glycol, to carry out reaction at atmospheric conditions, but the difficult-to-remove solvent stays with the surfactant.

A suitable method for preparing an N-acyl aminoalkane sulfonate surfactants as disclosed herein includes combining: (a) an aminoalkane sulfonic acid of formula (II) or (b) an anhydrous alkali salt of an aminoalkane sulfonic acid of formula (II):

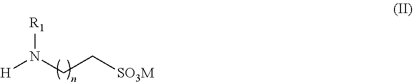

where R₁ represents H, or C₁ to C₄ alkyl radical, n is an integer from 1 to 2, and M is a cationic group selected from the group consisting of alkali metal salts and hydrogen, a waterless base, and a fatty alkyl ester of formula (III)

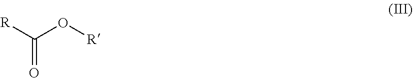

where R is selected from an C₅-C₂₁ alkyl substituent and R′ is a C₁ or higher alkyl substituent, preferably methyl, to form the mixture including N-acyl aminoalkane sulfonate of formula (I):

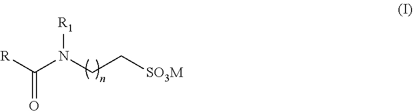

where R is an C₅-C₂₁ alkyl substituent, R₁ represents H, or C₁ to C₄ alkyl radical, n is an integer from 1 to 2, and M is a cationic group selected from the group consisting of alkali metal salts and hydrogen.

This process may prepare any of the surfactant compositions previously disclosed.

A reaction diagram for the formation of the surfactant composition is shown below:

### Amidation Reaction

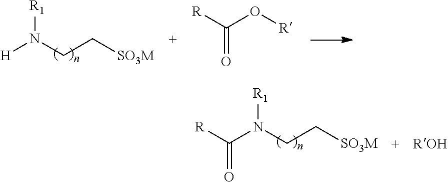

It was unexpectedly discovered that while making sodium N-acyl taurate surfactant via the following reaction:

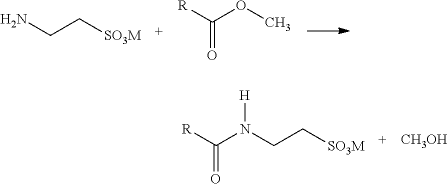

sodium N-acyl-N-methyl taurate surfactant was also formed as part of the surfactant composition:

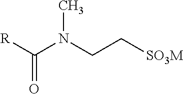

It is contemplated that under the reaction conditions the following side reaction occurs at temperatures greater than 150° C. to form sodium N-methyl taurine in-situ:

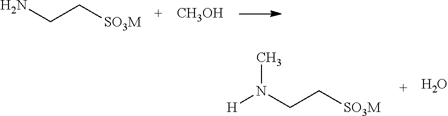

which then reacts with the fatty alkyl ester to form the useful and valuable co-product sodium N-acyl-N-methyl taurate surfactant, as shown in the reaction diagram below:

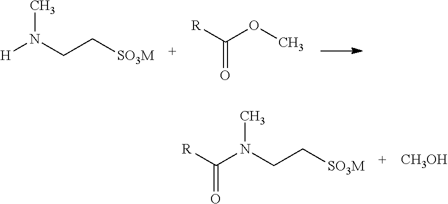

The combining step may include preparing a suspension of the aminoalkane sulfonic acid salt of formula (II) by adding the fatty alkyl ester of formula (III) to the anhydrous alkali salt of an aminoalkane sulfonic acid of formula (II), and contacting the suspension with the waterless base to form the mixture. It is desirable to add a solvent to the process because the amount of methanol being supplied by the catalytic amount of waterless base, sodium methoxide solution, or generated from the amidation reaction is not sufficient to overcome the lack of miscibility/compatibility between the said alkali metal salt of aminoalkane sulfonic acid and the fatty alkyl ester. The solvent may be the same or different from methanol, but it is preferable that is the same as present in the waterless base and the same as is being formed in the amidation reaction. Additionally or alternatively, the combining step may include combining the waterless base and the fatty alkyl ester of formula (III) to form a premixture and then adding (a) the aminoalkane sulfonic acid of formula (II) or (b) the anhydrous alkali salt of an aminoalkane sulfonic acid of formula (II) to the premixture to form the mixture. Additionally or alternatively, the combining step may include preparing a formulation of the aminoalkane sulfonic acid salt of formula (II) by adding the waterless base to the anhydrous alkali salt of an aminoalkane sulfonic acid of formula (II), and contacting the formulation with the fatty alkyl ester of formula (III) to form the mixture. Additionally or alternatively, the combining step may include preparing a formulation of the aminoalkane sulfonic acid salt of formula (II) by adding the waterless base to the aminoalkane sulfonic acid of formula (II), and contacting the formulation with the fatty alkyl ester of formula (III) to form the mixture.

A white, solid, agglomerate-looking material may form after (if) the aminoalkane sulfonic acid comes into contact with the waterless base only. Therefore, it is contemplated that lower conversions to and yields of N-acyl aminoalkane sulfonate surfactants may be achieved by adding aminoalkane sulfonic acid first to the waterless base (sodium methoxide solution) and adding the fatty alkyl ester afterwards. Thus, it is preferred in the process to form the alkali metal salt of an aminoalkane sulfonic acid in-situ by adding an aminoalkane sulfonic acid to mixture consisting of a waterless base and fatty alkyl ester. Additionally or alternatively, the process may include adding the waterless base to a suspension of the aminoalkane sulfonic acid in the fatty alkyl ester (FAME). Not to be bound by theory, this is advantageous because the resulting alkali metal salt of the aminoalkane sulfonic acid that forms is finely dispersed or soluble in the mixture containing fatty alkyl ester.

The process may include adding the N-acyl aminoalkane sulfonate surfactant to water to form a surfactant composition including greater than 20 wt. %, preferably greater than 25 wt. %, and more preferably greater than 30 wt. % N-acyl aminoalkane sulfonate surfactant by weight of the surfactant composition. The process may include adding the N-acyl aminoalkane sulfonate surfactant to water to form a surfactant composition including from 20 to 95 wt. %, from 20 to 90 wt. %, from 20 to 85 wt. %, from 20 to 80 wt. %, from 20 to 75 wt. %, from 20 to 70 wt. %, from 20 to 65 wt. %, from 20 to 60 wt. %, from 20 to 55 wt. %, from 20 to 50 wt. %, from 25 to 95 wt. %, from 25 to 90 wt. %, from 25 to 85 wt. %, from 25 to 80 wt. %, from 25 to 75 wt. %, from 25 to 70 wt. %, from 25 to 65 wt. %, from 25 to 60 wt. %, from 25 to 55 wt. %, from 25 to 50 wt. %, from 30 to 95 wt. %, from 30 to 90 wt. %, from 30 to 85 wt. %, from 30 to 80 wt. %, from 30 to 75 wt. %, from 30 to 70 wt. %, from 30 to 65 wt. %, from 30 to 60 wt. %, from 30 to 55 wt. %, from 30 to 50 wt. %, from 40 to 95 wt. %, from 40 to 90 wt. %, from 40 to 85 wt. %, from 40 to 80 wt. %, from 40 to 75 wt. %, from 40 to 70 wt. %, from 40 to 65 wt. %, from 40 to 60 wt. %, from 40 to 55 wt. %, from 40 to 50 wt. %, from 50 to 95 wt. %, from 50 to 90 wt. %, from 50 to 85 wt. %, from 50 to 80 wt. %, from 50 to 75 wt. %, from 50 to 70 wt. %, from 50 to 65 wt. %, from 50 to 60 wt. %, from 50 to 55 wt. %, from 55 to 95 wt. %, from 55 to 90 wt. %, from 55 to 85 wt. %, from 55 to 80 wt. %, from 55 to 75 wt. %, from 55 to 70 wt. %, from 55 to 65 wt. %, from 55 to 60 wt. %, from 60 to 95 wt. %, from 60 to 90 wt. %, from 60 to 85 wt. %, from 60 to 80 wt. %, from 60 to 75 wt. %, from 60 to 70 wt. %, from 60 to 65 wt. %, from 65 to 95 wt. %, from 65 to 90 wt. %, from 65 to 85 wt. %, from 65 to 80 wt. %, from 65 to 75 wt. %, from 65 to 70 wt. %, from 70 to 95 wt. %, from 70 to 90 wt. %, from 70 to 85 wt. %, from 70 to 80 wt. %, from 70 to 75 wt. %, from 75 to 95 wt. %, from 75 to 90 wt. %, from 75 to 85 wt. %, from 75 to 80 wt. %, from 80 to 95 wt. %, from 80 to 90 wt. %, from 80 to 85 wt. %, from 85 to 95 wt. %, from 85 to 90 wt. %, or from 90 to 95 wt. % N-acyl aminoalkane sulfonate surfactant by weight of the surfactant composition.

The process may include increasing the temperature of the mixture to 190° C. or less, preferably 170° C. or less, more preferably 160° C. or less to form a reaction mixture. The increasing step may include increasing the temperature of the mixture to from about 65° C. to about 190° C. or preferably from about 90° C. to about 160° C. The process may include continuously removing alkyl alcohol from the reaction mixture.

The (a) aminoalkane sulfonic acid of formula (II) may include taurine (2-aminocthanesulfonic acid), homotaurine (3-amino-1-propanesulfonic acid), N-methyl taurine (2-methylaminocthanesulfonic acid), or combinations thereof. The (b) the anhydrous alkali salt of an aminoalkane sulfonic acid of formula (II) may include sodium 2-aminoethanesulfonate, N-methyl taurine sodium salt, sodium 3-aminopropanesulfonate, sodium 3-(N-methylamino)propanesulfonate, and combinations thereof.

Suitable waterless bases for use are those selected from the group consisting of alkali metals, such as sodium, lithium and potassium: alloys of two or more alkali metals, such as sodium-lithium and sodium-potassium alloys; alkali metal hydrides, such as sodium, lithium and potassium hydride; and alkali metal alkoxides, especially those containing from about one to about four carbon atoms such as sodium methoxide, potassium methoxide, lithium methoxide, sodium ethoxide, potassium ethoxide, lithium ethoxide, sodium n-propoxide, potassium n-propoxide, sodium isopropoxide, potassium isopropoxide, sodium butoxide, potassium butoxide, sodium isobutoxide, potassium isobutoxide, sodium sec-butoxide, potassium sec-butoxide, and potassium tert-butoxide. Alkoxides are available in solid form or as solutions in the alcohol from which the alkoxide derives. The waterless base may include a C₁-C₄ alkoxide, preferably sodium methoxide, potassium methoxide in methanol solution, or combinations thereof.

The mixture may include from about 1.00 to about 1.50 moles, preferably from about 1.02 to about 1.20 moles, and more preferably from about 1.05 to about 1.10 moles of the waterless base per mole of (a) an aminoalkane sulfonic acid of formula (II). The mixture may include from about 0.01 to about 0.5 moles, preferably from about 0.02 to about 0.2 moles, and more preferably from about 0.05 to about 0.1 moles of the waterless base per mole of (b) an anhydrous alkali salt of an aminoalkane sulfonic acid of formula (II).

The relative molar amounts wherein the alkoxide is added to step i) in an amount within the range of 1.00 to 1.50 moles, 1.02 to 1.20 moles or 1.05 to 1.10 per mole of the amino acid, specifically reciting all values within these ranges and any ranges created thereby. The alkoxide not consumed in the neutralization catalyzes the reaction between amino acid salt and the fatty alkyl ester. Thus, in the process described herein the amount of alkoxide catalyst can range from 2 to 20 mole percent or from 5 to 10 mole percent, specifically reciting all values within these ranges and any ranges created thereby.

As used herein, the terms “fatty alkyl ester(s)” and “fatty acid esters” are intended to include any compound wherein the alcohol portion is easily removed, e.g. esters of volatile alcohols, C₁₋₄ alcohols (preferably methyl). Volatile alcohols are highly desirable. Methyl esters are the most highly preferred ester reactants. Suitable ester reactants can be prepared by the reaction of diazoalkanes and fatty acids or derived by alcoholysis from the fatty acids naturally occurring in fats and oils. Non-limiting examples are methyl octanoate (caprylate), methyl decanoate (caprate), methyl dodecanoate (laurate), methyl tetradecanoate (myristate), methyl hexadecanoate (palmitate), methyl octadecanoate (stearate), methyl oleate, ethyl dodecanoate (laurate), ethyl tetradecanoate (myristate), isopropyl dodecanoate (laurate), isopropyl tetradecanoate (myristate), and mixtures thereof. Suitable fatty acid esters can be derived from either synthetic or natural, saturated or unsaturated fatty acids. Non-limiting examples of saturated fatty acids include caprylic, capric, lauric, myristic, palmitic, and stearic. Mixtures of fatty acids derived from coconut oil, cottonseed oil, palm kernel oil, soybean oil, cotton seed oil, rapeseed oil, safflower oil, canola oil (low erucic acid), and corn oil and mixtures thereof. Most preferred is coconut oil.

It is preferred that the fatty alkyl esters be highly purified to remove color/odor materials, oxidation products, and their precursors. The free fatty acid level can be less than about 0.1% or less than about 0.05%, by weight of the esters. In addition, the fatty acid alkyl esters should have the lowest level of moisture possible, since any water present will react with the alkoxide catalyst, inhibit the amidation reaction and can lead to elevated levels of soap.

The process may include adding from about 0.90 to about 1.50 moles, preferably from about 0.95 to about 1.20 moles, or more preferably from about 1.00 to about 1.05 moles of the fatty alkyl ester per mole of the alkali salt of an aminoalkane sulfonic acid, specifically reciting all values within these ranges and any ranges created thereby. As shown in the examples, high active surfactant compositions with low levels of impurities are possible without further processing steps when the (a) aminoalkane sulfonic acid of formula (II) or (b) anhydrous alkali salt of an aminoalkane sulfonic acid of formula (II) and the fatty alkyl ester are used in about equimolar amounts. Using an excess of fatty alkyl ester would result in a surfactant composition contaminated with unreacted fatty alkyl ester, thus requiring further processing for its removal. It is even less desirable to use the (a) aminoalkane sulfonic acid of formula (II) or (b) anhydrous alkali salt of an aminoalkane sulfonic acid of formula (II) in excess since: (i) it is more expensive than the fatty alkyl ester, (ii) it does not have surface active properties and (iii) it would be difficult and costly to recover the unreacted amino acid salt from the surfactant mixture.

Surprisingly the reaction between the (a) aminoalkane sulfonic acid of formula (II) or (b) anhydrous alkali salt of an aminoalkane sulfonic acid of formula (II) and fatty alkyl ester of formula (III) can be performed at atmospheric or even under negative pressure while continuously distilling off alkyl alcohol (for example, methanol) from the reaction mixture. The temperature conditions for the amidation reaction may range from about 65° ° C. to about 190° C. or from about 90° C. to about 160° ° C., specifically reciting all values within these ranges and any ranges created thereby. Reaction progress can be monitored by tracking the amount of alkyl alcohol collected and/or by quantitative ¹H NMR, or other analytical techniques. The final high active N-acyl aminoalkane sulfonate surfactant reaction mixture, made under these conditions can be grinded, flaked, prilled, pelletized, and/or made into beads, noodles, needles, and ribbons by known methods to those skilled in the art.

The reaction may utilize an inert gas headspace to help reduce the level of oxygen available during the reaction. The reduced level of oxygen helps to reduce the amount of oxidation of the constituents of the reaction. Oxidation of the constituents can cause discoloration. A suitable example of an inert gas that may be utilized is nitrogen.

Additionally, the benefit of performing the reaction described herein at atmospheric or even negative pressure is that the resultant surfactant can be (if desired) substantially free of any solvents. Additionally, the alkyl alcohol, e.g. methanol, vapors can be condensed and recovered outside of the reactor. This collection of alkyl alcohol vapors can be re-used to make more methyl esters. The resultant surfactant can have less than about 5.0 wt % of fatty acid methyl ester, less than about 3.0 wt % or less than about 2.0 wt %, specifically reciting all values within these ranges and any ranges created thereby.

One of the advantages of the process of the present disclosure is that the resultant N-acyl aminoalkane sulfonate surfactant of formula (I) can be made to be substantially free of solvents, without using excesses of reactants, in high purity and without additional purifications steps.

In order to make a pumpable surfactant composition (pumpable at 50° C. or below), the active surfactant mixture without any further purification may be diluted into water in an amount of from 20 to 70 wt. percent of the surfactant mixture, and from about 25 to about 50 wt. percent of the surfactant mixture. Alternatively, the water may be added to the high active surfactant mixture at temperatures below 120° C. or under 100° C. under good mixing. The amount of water needed will depend on target surfactant active level, target viscosity and the solubility behavior of the surfactant. The solid form of the surfactant—powder, flakes, pellets, beads, needles, noodles—may also be dissolved in water to make a pumpable surfactant composition for formulators to easily incorporate in cleaning formulations.

The process may be carried out as batch, semicontinuous, or in a continuous mode using suitable reactor(s) configurations. A conventional stirred-tank batch reactor known by those skilled in the art equipped with a means for heating the reaction, a vapor column and condenser for collecting volatile alkyl alcohol, an efficient stirrer capable of stirring the reaction product mixture, a means for blanketing the reactor contents with nitrogen, and optionally a vacuum system capable of achieving a vacuum of less than 20 mm of Hg may be used to prepare the N-acyl aminoalkane sulfonate surfactant composition disclosed herein.

Other reactors useful in the present disclosure is appropriately an apparatus with which liquid and solid mixtures of liquid and solid substances can be mixed using shear forces. In a static housing, the movement of the reaction mixture are brought about by internal mechanical stirring or mixing devices. The reaction apparatus can be a kneader or mixer equipped with sigma blades, masticator blades, or plough type agitator. Additional useful apparatuses include horizontal or vertical forced mixers equipped with mixing tools, for example sigma blades, masticator blades, plough type agitator, or throwing paddles, in combination with a cutting rotor.

Suitable horizontal forced mixers are those equipped with mixing tools or combinations of mixing tools such as, for example, sigma blades, masticator blades, or plough type agitator, in combination with a cutting rotor installed in the drum; more preferably horizontal forced mixers operating at a Froude number between 0.1 and 6, between 0.25 and 5 or between 0.4 and 4, and equipped with mixing tools, or combinations of mixing tools, such as, for example sigma blades, masticator blades and plough type agitator in combination with a cutting rotor installed in in the drum. Without wishing to be bound by theory, in the treatment of mixing processes, the Froude number, Fr, plays a major role. This dimensionless quantity is indicative of the relationship between the forces of inertia and gravity acting on a moving particle. The following equation is applicable here:

Fr=v²/rg
where:
v=peripheral speed [m/s]
r=radius of mixing drum [m]
g=acceleration of gravity [m/s2]

v=π×D×n/60
where:
D=diameter of mixing drum [m]
N=rotation rate of shaft [rpm]

The N-acyl aminoalkane sulfonate surfactant composition and process for making described herein has a number of advantages over known commercial manufacturing processes and include:
1) High active surfactant composition substantially free of solvents and halide salts, like sodium chloride.
2) High conversion and yields can be achieved while avoiding laborious purification steps and concomitant product loss.
3) Fewer chemical engineering unit operations that can result in significant reduction in energy consumption.
4) Free of toxic and hazardous reagents, as described herein, and therefore the issue of handling these materials does not arise.
5) The resultant surfactant is substantially free of solvents that would need to be otherwise removed through additional post-reaction processing steps because they limit and/or impact the application and/or formulability of the surfactant.
6) Producing N-acyl aminoalkane sulfonate from fatty alkyl esters.
7) Conversion and yields of at least 75%, preferably 80% or greater.
8) Lower reaction temperatures.
9) Eliminates the need to prepare fatty acid chlorides.
10) Avoids the use of excess fatty acid (which in the end contaminates product and needs to be removed afterwards).
11) No waste streams, methanol recovered can be recycled to make fatty alkyl esters.
12) Reaction at atmospheric pressure (not pressurized conditions).

It is desirable to add a solvent to the process because the amount of methanol being supplied by the catalytic amount of waterless base, sodium methoxide solution, or generated from the amidation reaction is not sufficient to overcome the lack of miscibility/compatibility between the said alkali metal salt of aminoalkane sulfonic acid and the fatty alkyl ester. The solvent may be the same or different from methanol, but it is preferable that is the same as present in the waterless base and the same as is being formed in the amidation reaction.

A white, solid, agglomerate-looking material may form after the aminoalkane sulfonic acid comes into contact with the waterless base. Therefore, it is contemplated that lower conversions to and yields of N-acyl aminoalkane sulfonate surfactants may be achieved by adding aminoalkane sulfonic acid first to the waterless base (sodium methoxide solution) and adding the fatty alkyl ester afterwards. Thus, it is preferred in the process to form the alkali metal salt of an aminoalkane sulfonic acid in-situ by adding an aminoalkane sulfonic acid to mixture consisting of a waterless base and fatty alkyl ester. Not to be bound by theory, this is advantageous because the resulting alkali metal salt of the aminoalkane sulfonic acid that forms is finely disperse or soluble in the mixture containing fatty alkyl ester.

### Applications and Uses

In another aspect, the present disclosure is directed to a consumer product cleaning or personal care composition comprising about 0.001 wt. % to about 99.999 wt. % or about 0.1 wt % to about 80 wt. % of the N-acyl aminoalkane sulfonate surfactants as described herein, based on the total weight of the composition, and 0.001 wt. % to about 99.999 wt. % of one or more additional cleaning components, or one or more additional personal care components. In various embodiments, the at least one cleaning component is selected from the group consisting of a surfactant, an enzyme, a builder, an alkalinity system, an organic polymeric compound, a hueing dye, a bleaching compound, an alkanolamine, a soil suspension agent, an anti-redeposition agent, a corrosion inhibitor, and a mixture thereof. In some cases, the composition is selected from the group consisting of a granular detergent, a bar-form detergent, a liquid laundry detergent, a liquid hand dishwashing composition, a hard surface cleaner, a tablet, a disinfectant, an industrial cleaner, a highly compact liquid, a powder, and a decontaminant. In a class of cases, the composition is enclosed within a sachet or a multi compartment pouch comprising both solid and liquid compartments.

In some embodiments, the at least one personal care component is selected from the group consisting of an oil, and emollient, a moisturizer, a carrier, an extract, a vitamin, a mineral, an anti-aging compound, a surfactant, a solvent, a polymer, a preservative, an antimicrobial, a wax, a particle, a colorant, a dye, a fragrance, and mixtures thereof. In various cases, the composition is a shampoo, a hair conditioner, a hair treatment, a facial soap, a body wash, a body soap, a foam bath, a make-up remover, a skin care product, an acne control product, a deodorant, an antiperspirant, a shaving aid, a cosmetic, a depilatory, a fragrance, and a mixture thereof. In a class of cases, the composition is delivered in a form selected from the group consisting of a wipe, a cloth, a bar, a liquid, a powder, a creme, a lotion, a spray, an aerosol, a foam, a mousse, a serum, a capsule, a gel, an emulsion, a doe foot, a roll-on applicator, a stick, a sponge, an ointment, a paste, an emulsion spray, a tonic, a cosmetic, and mixtures thereof. In various embodiments, the composition further comprises a product selected from the group consisting of a device, an appliance, an applicator, an implement, a comb, a brush, a Substrate, and mixtures thereof. In some embodiments, the composition is dispensed from an article selected from the group consisting of a bottle, a jar, a tube, a sachet, a pouch, a container, a tottle, a vial, an ampoule, a compact, a wipe, and mixtures thereof.

### Further Process Embodiments

In embodiments, the process may include an amidation reactor. In embodiments, an aminoalkane sulfonic acid stream, a waterless base stream, and a fatty alkyl ester stream are fed to amidation reactor which produces a first stream having N-acyl aminoalkane sulfonate surfactant and a second stream having alkyl alcohol vapor with entrained fatty alkyl ester. In embodiments, there may be an addition of an alkyl alcohol recovery step which separates any fatty alkyl ester entrained in the second stream (the alkyl alcohol vapor stream) to form a third stream (fatty alkyl ester stream) and recovers the alkyl alcohol and producing a fourth stream which is recovered alkyl alcohol that can be used as is, or optionally after further purification, in a separate process to make fatty alkyl esters or for other uses. The third stream having a composition of fatty alkyl ester and alkyl alcohol can be optionally further purified.

In embodiments, a solid handling (cooling-breaking/grinding) unit may be added. In this embodiment, the first stream (N-acyl aminoalkane sulfonate surfactant) is then fed to the solid handling unit to form a N-acyl aminoalkane sulfonate surfactant product stream. In embodiments, the N-acyl aminoalkane sulfonate surfactant product stream is no longer hot and is substantially free of impurities including water, salt (NaCl), polyol solvents, alkyl alcohol.

In embodiments, a dissolution unit/reactor may be added. In this embodiment, the first stream (N-acyl aminoalkane sulfonate surfactant) is fed to a dissolution unit to produce a N-acyl aminoalkane sulfonate surfactant aqueous solution stream.

In embodiments, a second amidation reactor may be added. In embodiments, the second amidation reactor may operate in parallel or in sequence with the first amidation reactor. In embodiments where the second amidation reactor operates in parallel with the first amidation reactor, the second amidation reactor produces a fifth stream having alkyl alcohol vapor with entrained fatty alkyl ester. The fifth stream may be fed to the alkyl alcohol recovery unit after combining with the alkyl alcohol vapor stream from the first amidation reactor. Without intending to be bound by theory, adding the second amidation reactor provides a convenient and effective way to increase product output, schedule reactor maintenance and/or repairs without increasing manufacturing plant footprint and capital expenditure to install a second alkyl alcohol recovery unit and utilities (hot oil boiler system).

In embodiments, the reaction surfactant product might contain unreacted fatty acid esters, unreacted taurine salt, fatty acid soap or other low-level impurities. Since one or more of these impurities may be undesirable in certain specific applications because of a potential negative impact, for example taste in toothpaste, efforts were made to develop post-reaction steps to purify the surfactant.

In an embodiment, the resultant reaction product containing N-acyl aminoalkane sulfonate surfactant is preferably dissolved in a mixture containing a C1-C4 aliphatic alcohol and water, heated to reflux, then cooled down to precipitate or crystallize the N-acyl aminoalkane sulfonate surfactant which is recovered by filtration. The C1-C4 aliphatic alcohol may be linear or isomeric butanol, propanol, ethanol or methanol or mixtures thereof.

The purification step comprises adding crude N-acyl aminoalkane sulfonate reaction mixture to an aqueous-aliphatic alcohol solvent medium comprising of about 5-25% water by weight and 95-75% aliphatic alcohol by weight. The resultant slurry mixture is heated to a temperature until fully solubilizing solid added, then the solution is cooled to ambient temperature or lower, 5-15° C., to precipitate (or crystallize) higher purity N-acyl aminoalkane sulfonate surfactant while the impurities stay in solution, and then filtering solid to obtain higher purity surfactant. The solids content comprises 7-20% by weight of the resultant slurry mixture upon adding crude N-acyl aminoalkane sulfonate reaction to aqueous-aliphatic alcohol solvent medium.

The high purity surfactant obtained by this purification step may be further purified if required. Example 15 below was effective and should not be regarded as limitative, it only represents one of many preferred embodiments.

### EXAMPLES

Analysis via chromatography/mass spectrometry revealed/confirmed the presence of N-acyl-N-methylaminoalkane sulfonate in the resultant product from reactions to N-acyl aminoalkane sulfonates. Samples were weighed and diluted with 100% MeOH to a concentration of 1 mg/mL (assuming 100% purity). Solutions were further diluted to 50 ppm with 50/50 MeOH/Water. Samples analyzed by UPLC-CAD-HRMS on a reverse phase ACQUITY UPLC BEH C18 2.1×150 mm (5 μL injection). Full scan and MS2 scan information were collected in both negative-ion electrospray mode.

Further analysis of samples via 2DNMR experiments further confirmed the presence of N-acyl-N-methylaminoalkane sulfonates and NMR peaks assignments made.

Analysis of the reaction products were conducted by a 1H NMR method.

In a scintillation vial reaction product and an internal standard (IS) were weighed out in a precision balance (0.1 mg readability). A 2:1 v/v solvent mixture of deuterated chloroform-methanol (CDCl₃—CD₃OD) was added to the vial to fully dissolve sample and IS (sometimes a drop or two of D₂O was required to fully dissolve sample). The quantitative 1H NMR spectra were recorded at 600 MHz using standard 1H pulse sequence, pulse width of 12.00, 60 sec delay, and a 2.59 sec acquisition time. NMR data was processed using MestReNova software version 14.2.1. The integration of the triplet at δ 3.54-3.56 ppm assigned to the methylene (—CH₂—) group of the N-acyl aminoalkane sulfonate surfactant was used to calculate the wt. %. The integration of the peaks at δ 3.73-3.76 & 3.68-3.71 ppm corresponding to rotamers of the methylene (—CH₂—) group of N-acyl-N-methylaminoalkane sulfonate surfactant was used to calculate the wt. %. The integration of a singlet at δ 3.65 ppm assigned to the methyl (CH₃—) of any residual fatty methyl ester was used to calculate the wt. %. The integrations were compared to the integration region of the IS and used for the calculations. The wt. % of each species was calculated using the following equation:

$$Wt . \% ( y ) = \frac{A ( y )}{A ( IS )} \times \frac{n ( IS )}{n ( y )} \times \frac{M W ( y )}{M W ( IS )} \times \frac{W ( IS )}{W ( y )} \times P ( IS )$$

Wt. % (y)=weight percent of “y” species in the sample
A=NMR integration
n=number of protons
MW=molecular weight
P=purity of the internal standard

The same procedure was repeated but instead the sample and IS were dissolved in deuterium oxide (D₂O). The integration of the triplet at δ 3.56-3.59 ppm assigned to the methylene (—CH₂—) group of N-acyl aminoalkane sulfonate surfactant was used to calculate the wt. %. And the integration of the triplet at δ 2.16-2.18 ppm for the methylene (—CH₂—C(O)—OM) adjacent to the carboxylate group was used to calculate the wt. % of fatty acid soap. The soap level could not be quantified in the deuterated chloroform-methanol (CDCl₃—CD₃OD) solvent system because the soap peak (—CH₂—C(O)—OM) partially overlaps with another peak (—CH₂—C(O)—NH—) corresponding to the surfactant, but it does not in D₂O.

Equipment: A horizontal forced mixer equipped with plough type agitator was used to carry out the transformation. It was equipped with a thermocouple mounted in the mixing drum with a digital temperature read out, a heating jacket of labyrinth design to ensure uniform flow around the mixing drum, a condenser adapted to a cover affixed to a flanged port on top of the vessel, receiver on a weighing balance, and an inert gas inlet. A discharge port using a manual ball valve is available at the bottom of the mixing drum. The mixer was heated using a heating circulator with heating fluid. The reaction of the product was unloaded unto glass baking trays The amount of methanol condensed (grams), the temperature of the reaction mixture (C) and the inlet temperature (° C.) of the heating fluid were trended in real time.

### Raw Material Used:

2-aminoethanesulfonic acid (Taurine), crystalline solid
Sodium methoxide solution—about 25 wt. % in methanol
CE-1270—a Lauric/Myristic 75/25 blend of C₁₂ and C₁₄ methyl esters
Coco fatty acid methyl ester (Coco FAME)—carbon-chain length distribution range for coco fatty acid methyl esters are shown below:
C₈=0-10%
C₁₀=0-7%
C₁₂=50-65%
C₁₄=18-22%
C₁₆=0-10%
C₁₈=0-10%
C₁₈₋₁=0-7%

### Examples 1-4

### Preparation of Sodium C1214 Taurate

Reactor was charged with CE-1270 (769.0 g, 3.46 mol), sodium methoxide (790.8 g, 3.53 mol) and taurine (413.0 g, 3.30 mol) under nitrogen and mixing at a temperature of 22-35° C. The temperature of the reaction mixture was gradually increased. The mixer was operated at a Froude number between 0.4 and 2 depending on the rheology of the composition. The temperature of the reaction mixture was gradually increased. Methanol started to distill off and was condensed when the reaction mixture temperature reached 68-69° C. and held steady for a period. The temperature of the reaction mixture began steadily to climb when about ≥60% of methanol of the total theoretical amount of methanol expected had been collected. The contents in the reactor were heated to 168° C. The reaction mixture was held from 165-168° C. for 110 min. Methanol from the base, and formed during the reaction, distilled off and condensed as the temperature climbed. The mixer and its contents were then cooled to ambient temperature while the shaft with plough mixing elements continued to rotate. A powdery product was unloaded from the mixing drum through the bottom port after opening the ball valve. Additional product was collected manually after removing the front cover bolted to the mixing drum. A yield of 1100 g was collected of an off-white solid powder.

Other experiments were conducted to evaluate the effect of temperature and time, they are summarized in Table A. Starting materials and conditions are identical to experiment 1, except where otherwise noted through footnotes.

TABLE A

| Example # | 1 | 2 | 3 | 4 |
|---|---|---|---|---|
| FAME | CE-1270 | CE-1270 | CE-1270 | C₈ to C₁₆ |
| | | | | FAME^{a} |
| Reaction Temperature (° C.) | 165-168 | 154-157 | 186-189 | 164-167 |
| Time at Temperature (min.) | 110 | 180 | 180 | 70 |
| Product Composition | | | | |
| Taurate Surf. (wt. %) | 76.4 | 73.7 | 75.5 | 76.8 |
| N-NMT Surf. (wt. %) | 8.1 | 7.1 | 6.0 | 6.8 |
| Soap (wt. %) | 8.4 | 8.7 | 9.4 | 8.5 |
| FAME (wt. %) | 1.9 | 3.6 | 3.9 | 1.5 |
| Na-Taurine (wt. %) | n/a | n/a | n/a | n/a |
| Results | | | | |
| Product Yield (g) | 1100 | 1092.6 | 1114.5 | 1063.6 |
| Conversion (%, | 83.2 | 79.0 | 81.3 | 81.8 |
| based on Taurine) | | | | |
| Taurate Reaction Yield (%)^{b} | 90.8 | 91.5 | 92.9 | 92.2 |
| Overall Taurate Yield (%)^{c} | 75.5 | 72.3 | 75.6 | 75.4 |
| N-MMT Yield (%)^{c} | 8.0 | 7.0 | 6.0 | 6.7 |
| Total Surfactant Yield (%)^{c} | 83.5 | 79.3 | 81.6 | 82.0 |
| APHA^{b} | 65 | 341 | 401 | 111 |
| Gardner^{b} | 3.5 | 9.0 | 10 | 4.9 |

^{a}C-chain length distribution: C₈ = 10.2%, C₁₀ = 7.4%, C₁₂ = 57.4%, C₁₄ = 20.6%, C₁₆ = 4.1%; NMT = N-Methyl Taurate; n/a = not available
^{b}calculated as the quantity of moles of taurate surfactant formed in relation to the moles of taurine consumed.
^{c}calculated as the mass of taurate surfactant formed in relation to the total maximum weight of taurate surfactant that could be produced.
^{b}10 wt. % surfactant solution in deionized water, LovibondÒ PFX-i Series, S/N 104146, 100 mm cell path.

As shown in Table A, example 2 shows that at a 10-degree drop in temperature vs example 1 leads to a slightly lower conversion and yields despite extending the time at this temperature. Similar conversion and yields are achieved in example 3 with a 20-degree increase in temperature & 70 min increase in time vs example 1, however the product comparatively exhibits more color development as shown by the APHA and Gardner values. Example 4 demonstrates that the reaction yields a product of similar quality at the same temperature but shorter time vs example 1 using a FAME containing C₈ and C₁₀ carbon chains in the composition. These experiments show that temperature and time at temperature are critical parameters in achieving good conversions and yields, and there is an optimum range to do it with low color development.

### Examples 5-8

The carbon-chain length distribution of the Coco FAME used in this experiment: C₈=8.6%, C₁₀=6.2%, C₁₂=50.1%, C₁₄=18.0, C₁₆=8.8% and C_{18:0}=8.1%.

### Preparation of Sodium Cocoyl Taurate

Reactor was charged with Coco FAME (768.5 g, 3.50 mol), sodium methoxide (839.5 g, 3.75 mol) and taurine (438.2 g, 3.50 mol) under nitrogen and mixing at a temperature of 22-35° C. The temperature of the reaction mixture was gradually increased. The mixer was operated at a Froude number between 0.4 and 2 depending on the rheology of the composition. The temperature of the reaction mixture was gradually increased. Methanol started to distill off and was condensed when the reaction mixture temperature reached 68-69° C. and held steady for a period. The temperature of the reaction mixture began steadily to climb when about ≥60% of methanol of the total theoretical amount of methanol expected had been collected. The contents in the reactor were heated to 191° C. The reaction mixture was held from 188-191° C. for 120 min. Methanol from the base, and formed during the reaction, distilled off and condensed as the temperature climbed. The mixer and its contents were then cooled to ambient temperature while the shaft with plough mixing elements continued to rotate. A powdery product was unloaded from the mixing drum through the bottom port after opening the ball valve. Additional product was collected manually after removing the front cover bolted to the mixing drum.

The powdered product contained 75.0 wt. % sodium cocoyl taurate, 7.3 wt. % sodium cocoyl N-methyl taurate, 9.1 wt. % fatty acid soap, 0.5 wt. % FAME. A solution of this surfactant (active surfactant 10 wt. %) exhibited a Gardner value=4.7 and APHA=104 measured in a Lovibond PFX-i Series, S/N 104146, 100 mm cell path.

Additional experiments were executed to evaluate using a coco FAME containing unsaturated C-chains. They are summarized in Table B. The FAME feedstock used had a slight yellow hue when compared to the fully saturated version used in example 5. Reactants and conditions were identical to example 5, except where otherwise noted through footnotes. Examples 6 and 8 produced respectively sodium cocoyl taurate surfactant compositions of similar quality to the fully saturated version in example 5. However, it can be observed that the higher temperature and time in example 8 leads to an increased in color development. On the other hand, example 7 shows that the time at temperature was not sufficiently long enough to convert the reactants into products as can be observed by the relatively high level of FAME in the surfactant composition.

TABLE B

| Example # | 6 | 7 | 8 |
|---|---|---|---|
| FAME^{a} | C₈-C_{18:1} | C₈-C_{18:1} | C₈-C_{18:1} |
| Reaction Temperature (° C.) | 169-172 | 181-184 | 188-191 |
| Time at Temperature (min.) | 90 | 55 | 150 |
| Product Composition | | | |
| Taurate Surf. (wt. %) | 78.1 | 67.9 | 76.0 |
| N-NMT Surf. (wt. %) | 7.2 | n/a | 7.4 |
| Soap (wt. %) | 9.0 | 7.7 | 9.9 |
| FAME (wt. %) | 1.3 | 11.1 | 0.9 |
| Na-Taurine (wt. %) | n/a | n/a | n/a |
| APHA^{b} | 144 | n/a | 222 |
| Gardner^{b} | 5.6 | n/a | 6.8 |

^{a}C-chain length distribution: C₈ = 8-8.6%, C₁₀ = 5.7-6.2%, C₁₂ = 50.0%, C₁₄ = 18.0%, C₁₆ = 9-10%, C_{18:0} = 0.9%, C_{18:1} = 6.5-6.7%
^{b}10 wt. % surfactant solution in deionized water, Lovibond ® PFX-i Series, S/N 104146, 100 mm cell path.
NMT = N-Methyl Taurate; n/a = not available

### Example 9

The carbon-chain length distribution of the FAME used in this experiment: C₁₂=59.3%, C₁₄=21.3, C₁₆=9.1% and C_{18:0}=1.3%, C_{18:1}=8.2%, C_{18:2}=0.6%.

Preparation of Sodium C₁₂-C₁₈ Taurate

Reactor was charged with FAME (802.9 g, 3.47 mol), sodium methoxide (795.3 g, 3.53 mol) and taurine (413.0 g, 3.30 mol) under nitrogen and mixing at a temperature of 22-35° C. The temperature of the reaction mixture was gradually increased. The mixer was operated at a Froude number between 0.4 and 2 depending on the rheology of the composition. The temperature of the reaction mixture was gradually increased. Methanol started to distill off and was condensed when the reaction mixture temperature reached 70-71° C. and held steady for a period. The temperature of the reaction mixture began steadily to climb when about ≥60% of methanol of the total theoretical amount of methanol expected had been collected. The contents in the reactor were heated to 172° C. The reaction mixture was held from 169-172° C. for 210 min. Methanol from the base, and formed during the reaction, distilled off and condensed as the temperature climbed. The mixer and its contents were then cooled to ambient temperature while the shaft with plough mixing elements continued to rotate. A light yellow “granular” product was unloaded from the mixing drum through the bottom port after opening the ball valve. Additional product was collected manually after removing the front cover bolted to the mixing drum.

The powdered product contained 75.5 wt. % sodium C1218 taurate, 6.7 wt. % sodium N-methyl C1218 taurate, 9.6 wt. % fatty acid soap, 2.2 wt. % FAME. A solution of this surfactant (active surfactant 10 wt. %) exhibited a Gardner value=11.3 and APHA>505 measured in a Lovibond PFX-i Series, S/N 104146, 100 mm cell path.

### Example 10

The carbon-chain length distribution of the FAME used in this experiment: C₁₂=60.4%, C₁₄=21.7, C₁₆=8.5% and C_{18:0}=1.0%, C_{18:1}=7.4%, C_{18:2}=0.5%.

Preparation of Sodium N-Methyl C₁₂-C₁₈ Taurate

Reactor was charged with FAME (809.2 g, 3.50 mol), sodium methoxide (59.4 g, 0.25 mol), 250 mL of methanol and dry sodium N-methyl taurine (576.6 g, 3.58 mol) under nitrogen and mixing at a temperature of 22-35° C. The temperature of the reaction mixture was gradually increased. The mixer was operated at a Froude number between 0.4 and 2 depending on the rheology of the composition. The temperature of the reaction mixture was gradually increased. Methanol started to distill off and was condensed when the reaction mixture temperature reached 74° C. and began steadily to climb. The contents in the reactor were heated to 182° C. The reaction mixture was held from 179-182° C. for 70 min. All the methanol distilled off and condensed as the temperature climbed. The mixer and its contents were then cooled to ambient temperature while the shaft with plough mixing elements continued to rotate. An off-white reaction product was unloaded manually after removing the front cover bolted to the mixing drum. It contained 55.0 wt. % sodium N-methyl C1218 taurate, 10.3 wt. % fatty acid soap, 13.6 wt. % FAME, and undetermined level of sodium N-methyl taurine.

### Example 11

### Comparative Example

### Impact of Added Glycerine

This example illustrates that the presence of glycerine as solvent leads to significantly lower conversion of taurine and yield of the surfactant.

The carbon-chain length distribution of the FAME used in this experiment: C₈=6.4%, C₁₀=64.9%, C₁₂=62.9%, C₁₄=22.6, C₁₆=2.6%.

Preparation of Sodium C₈-C₁₆ Taurate

Reactor was charged with FAME (1148.2 g, 5.36 mol), sodium methoxide (1220.4 g, 5.74 mol), glycerine (177.1 g) and taurine (670.9 g, 5.36 mol) under nitrogen and mixing at a temperature of 22-35° C. The temperature of the reaction mixture was gradually increased. The mixer was operated at a Froude number between 0.4 and 2 depending on the rheology of the composition. The temperature of the reaction mixture was gradually increased. Methanol started to distill off and was condensed when the reaction mixture temperature reached 68-69° C. and held steady for a period. The contents in the reactor were heated to 157° C. The reaction mixture was held from 155-157° C. for 120 min. Methanol from the base, and formed during the reaction, distilled off and condensed as the temperature climbed. The mixer and its contents were then cooled to ambient temperature while the shaft with plough mixing elements continued to rotate. A pasty reaction product with a heavy fatty acid methyl ester smell was unloaded manually from the reactor after removing the front cover bolted to the mixing drum. It contained 24.6 wt. % sodium C8-C16 taurate, 5.6 wt. % fatty acid soap, 21.8 wt. % FAME, and 26.3 wt. % sodium taurine salt.

### Example 12

### 130 L Reactor Size

A 130-liter horizontal forced mixer (FM-130 Plow Batch Mixer available from B&P Littleford) equipped with plough type agitator and high temperature heating jacket (hot oil) mounted on an industrial digital floor scale and equipped with two-condenser and two-receiver system, and an inert gas inlet, was charged with fatty acid methyl ester CE1270 (Methyl Laurate/Myristate available from P&G Chemicals) (29.0 kg), sodium methoxide solution, 25 wt. % in methanol available from Sigma-Aldrich (29.1 kg), and 2-aminoethanesulfonic acid (Taurine available from Spectrum Chemical Mfg. Corp.) (15.7 kg) under nitrogen. The mixture was gradually brought to 150° C. over 10 hrs, during which period the methanol evaporated was condensed outside the mixer. Any fatty acid methyl ester entrained in the methanol vapor was condensed (˜70-80° C. condenser) & collected in first receiver, while the methanol was condensed (˜5-10° C. condenser) & collected in second receiver. The reaction mixture was kept between 150-160° C. for 2 hrs. The methanol collected was 30.0 kg. The mixer and the product mass in it were then cooled to ambient temperature while the shaft with plough mixing elements continued to rotate. A powdery product flowed freely and was unloaded from the mixer into a lined fiber drum through the discharged port at the bottom of the vessel. A yield of 42.6 kg was collected. The final product had the following composition determined via NMR analysis: 74.15 wt. % sodium C1214 taurate, 7.05 wt. % sodium C1214 N-methyl taurate, 7.80 wt. % fatty acid soap, 4.75 wt. % FAME. A solution of this surfactant (active surfactant 10 wt. %) exhibited a Gardner value=0.3 and APHA=68.5 measured in a Lovibond PFX-i Series, S/N 104146, in 10 mm & 100 mm cell path respectively. A Magnetic Resonance Spectrometer, Bruker Avance III 600 MHz w/SampleJet was used for NMR analysis. The NMR data was processed using MestReNova software version 14.2.1 available from Mestrelab.

### Example 13

### 130 L Reactor Size

A 130-liter horizontal forced mixer (FM-130 Plow Batch Mixer available from B&P Littleford) equipped with plough type agitator and high temperature heating jacket (hot oil) mounted on an industrial digital floor scale and equipped with two-condenser and two-receiver system, and an inert gas inlet, was charged with fatty acid methyl ester CE1270 (Methyl Laurate/Myristate available from P&G Chemicals) (36.2 kg), sodium methoxide solution, 25 wt. % in methanol available from Sigma-Aldrich (36.1 kg), and 2-aminocthanesulfonic acid (Taurine available from Spectrum Chemical Mfg. Corp.) (19.5 kg) under nitrogen. The mixture was gradually brought to 150° C. over 7 hrs, during which period the methanol evaporated was condensed outside the mixer. Any fatty acid methyl ester entrained in the methanol vapor was condensed (˜70-80° C. condenser) & collected in first receiver, while the methanol was condensed (˜5-10° C. condenser) & collected in second receiver. The reaction mixture was kept between 150-160° C. for 2.5 hrs. The methanol collected was 37.0 kg.

The mixer and the product mass in it were then cooled to ambient temperature while the shaft with plough mixing elements continued to rotate. A powdery product flowed freely and was unloaded from the mixer into a lined fiber drum through the discharged port at the bottom of the vessel. A yield of 53.7 kg was collected. The final product had the following analysis: 72.40 wt. % sodium C1214 taurate, 9.10 wt. % sodium C1214 N-methyl taurate, 8.85 wt. % fatty acid soap, 3.80 wt. % FAME. A solution of this surfactant (active surfactant 10 wt. %) exhibited a Gardner value=0.4 and APHA=77.3 measured in a Lovibond PFX-i Series, S/N 104146, in 10 mm & 100 mm cell path respectively. A Magnetic Resonance Spectrometer, Bruker Avance III 600 MHz w/SampleJet was used for NMR analysis. The NMR data was processed using MestReNova software version 14.2.1 available from Mestrelab.

A 130-liter horizontal forced mixer (FM-130 Plow Batch Mixer available from B&P Littleford) equipped with plough type agitator and high temperature heating jacket (hot oil) mounted on an industrial digital floor scale and equipped with two-condenser and two-receiver system, and an inert gas inlet, was charged with fatty acid methyl ester CE1270 (Methyl Laurate/Myristate available from P&G Chemicals) (41.2 kg), sodium methoxide solution, 25 wt. % in methanol available from Sigma-Aldrich (40.9 kg) and 2-aminoethanesulfonic acid (Taurine available from Spectrum Chemical Mfg. Corp.) (22.2 kg) under nitrogen. The mixture was gradually brought to 150° C. over 6.5 hrs, during which period the methanol evaporated was condensed outside the mixer. Any fatty acid methyl ester entrained in the methanol vapor was condensed (˜70-80° C. condenser) & collected in first receiver, while the methanol was condensed (˜5-10° C. condenser) & collected in second receiver. The reaction mixture was kept between 150-160° C. for 3.5 hrs. The hot reaction product mass was then discharged into a vessel located beneath the mixer. The vessel contained water at ambient temperature and was equipped with an impeller. The top of the vessel was covered by an enclosure that connected it to the contour discharge opening located at the bottom of the mixer. A N₂ gas stream flowed through the system to displace any air and to blanket the discharge system with N₂. The hot reaction product mass was transferred from the mixer by opening discharge port door allowing the material to drop into the water-containing vessel while mixing with impeller to dissolve & cool surfactant resulting in a 61° C. concentrated surfactant aqueous solution.

The mixer was relatively clean, there was about 100 g of residual solid product inside after it was inspected once cool. This residual solid was grinded and analyzed: 70.2 wt. % sodium C1214 taurate, 4.3 wt. % sodium C1214 N-methyl taurate, 8.1 wt. % fatty acid soap, 8.3 wt. % FAME. A solution of this surfactant (active surfactant 10 wt. %) exhibited a Gardner value=0.3 and APHA=58.9 measured in a Lovibond PFX-i Series, S/N 104146, in 10 mm & 100 mm cell path respectively. A Magnetic Resonance Spectrometer, Bruker Avance III 600 MHz w/SampleJet was used for NMR analysis. The NMR data was processed using MestReNova software version 14.2.1 available from Mestrelab.

### Example 15

### I. Preparation and Purification of Sodium C1214 Taurate

A 22-liter horizontal forced mixer (MDVT-22 Plow Batch Mixer available from B&P Littleford) equipped with plough type agitator and high temperature heating jacket (hot oil), equipped with a condenser and receiver, and an inert gas inlet, was charged with fatty acid methyl ester CE1270 (Methyl Laurate/Myristate available from P&G Chemicals) (7.01 kg), sodium methoxide solution, 25 wt. % in methanol available from Sigma-Aldrich (6.99 kg) and 2-aminoethanesulfonic acid (Taurine available from Spectrum Chemical Mfg. Corp.) (3.75 kg) under nitrogen. The mixture was gradually brought to 150° C. over 8 hrs, during which period the methanol evaporated was condensed outside the mixer. The reaction mixture was kept between 150-168° C. for 6 hrs. The mixer and the product mass in it were then cooled to ambient temperature while the shaft with plough mixing elements continued to rotate. A powdery product flowed freely and was unloaded from the mixer through a port at the bottom of the vessel. The final product had the following analysis: 69.8 wt. % sodium C1214 taurate, 5.0 wt. % sodium C1214 N-methyl taurate, 8.4 wt. % fatty acid soap, 8.4 wt. % FAME. A Magnetic Resonance Spectrometer, Bruker Avance III 600 MHz w/SampleJet was used for NMR analysis. The NMR data was processed using MestReNova software version 14.2.1 available from Mestrelab.

### II. Purification of Sodium C1214 Taurate

To a 2.5 L vessel equipped with overhead stirrer and heating unit was added 1200 g of an ethanol-water solvent mixture consisting of 10% of water by weight. 100 g of powdered reaction product as obtained above was added to the solvent mixture while mixing, and then the temperature of the resultant slurry mixture was increased until the solid completely dissolved yielding a clear solution. The mixture was then cooled down to ambient temperature. The precipitate formed was recovered by vacuum-assisted filtration using a Buchner funnel apparatus and filter paper (Whatman® 40). The solid cake was then dried. The dry purified product had the following analysis: 92.1 wt. % sodium C1214 taurate, 2.0 wt. % sodium C1214 N-methyl taurate, 5.1 wt. % fatty acid soap, 0.2 wt. % FAME. A Magnetic Resonance Spectrometer, Bruker Avance III 600 MHZ w/SampleJet was used for NMR analysis. The NMR data was processed using MestReNova software version 14.2.1 available from Mestrelab.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any subject matter disclosed or claimed herein or that it alone, or in any combination with any other reference or references, teaches, suggests or discloses any such subject matter. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A surfactant composition comprising:
greater than 75 wt. % N-acyl aminoalkane sulfonate of formula (I), by weight of the surfactant composition: 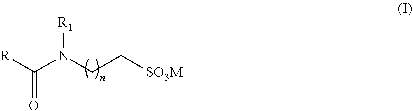
wherein:
R is an C₅-C₂₁ alkyl substituent, R₁ represents H, or C₁ to C₄ alkyl radical, n is an integer from 1 to 2, and M is a cationic group selected from the group consisting of alkali metal salts and hydrogen, and
the surfactant composition is substantially free of solvent and NaCl.

2. The composition of claim 1, further comprising at least 5 wt. %, preferably from about 5 to about 15 wt. %, more preferably about 8 to about 10 wt. % N-acyl-N-methylaminoalkane sulfonate surfactant by weight of the surfactant composition.

3. The surfactant composition of claim 1, wherein the alkyl substituent is saturated.

4. The surfactant composition of claim 1, wherein the alkyl substituent is unbranched.

5. The surfactant composition of claim 1, wherein R is a C₇-17 alkyl substituent.

6. The surfactant composition of claim 1, wherein the surfactant composition is substantially free of polyol solvents and water.

7. The surfactant composition of claim 1, comprising greater than 85 wt. % of the N-acyl aminoalkane sulfonate surfactant, by weight of the surfactant composition.

8. The surfactant composition of claim 1, further comprising from 0 to 1 wt. % of an alkyl alcohol (R′OH), by weight of the composition.

9. The surfactant composition of claim 1, comprising less than about 5%, or more preferably less than about 3% of fatty acid methyl ester by weight of the surfactant composition.

10. The surfactant composition of claim 1, wherein the surfactant composition is selected from the group consisting of a powder, granule, flake, noodle, needle, extrudate, ribbon, bead and pellet and mixtures thereof.

11. The surfactant composition of claim 1, wherein said surfactant composition is a form selected from the group consisting of a granular detergent, a bar-form detergent, a liquid laundry detergent, a gel detergent, a single-phase or multi-phase unit dose detergent, a detergent contained in a single-phase or multi-phase or multi-compartment water soluble pouch, a liquid hand dishwashing composition, a laundry pretreat product, a surfactant contained on or in a porous substrate or nonwoven sheet, an automatic dish-washing detergent, a hard surface cleaner, a fabric softener composition, a personal care composition and mixtures thereof.

12. A process for preparation of a surfactant composition comprising a N-acyl aminoalkane sulfonate surfactant comprising:
combining:
(a) an aminoalkane sulfonic acid of formula (II) or (b) an anhydrous alkali salt of an aminoalkane sulfonic acid of formula (II): 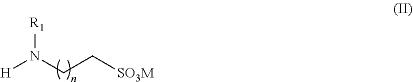
wherein R₁ represents H, or C₁ to C₄ alkyl radical, n is an integer from 1 to 2, and M is a cationic group selected from the group consisting of alkali metal salts and hydrogen,
a waterless base, and
a fatty alkyl ester of formula (III) [symbol]
(III)
wherein R is selected from an C₅-C₂₁ alkyl substituent and R′ is a C₁ or higher alkyl substituent, preferably methyl, to form the mixture comprising:
N-acyl aminoalkane sulfonate of formula (I): 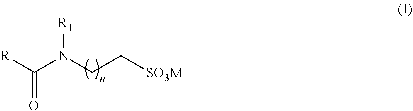
wherein:
R is an C₅-C₂₁ alkyl substituent, R₁ represents H, or C₁ to C₄ alkyl radical, n is an integer from 1 to 2, and M is a cationic group selected from the group consisting of alkali metal salts and hydrogen;
increasing the temperature of the mixture to 190° C. or less, preferably 170° C. or less, more preferably 160° C. or less to form a reaction mixture; and
continuously removing alkyl alcohol from the reaction mixture.

13. The process of claim 12, wherein the surfactant composition further comprises at least 5 wt. %, preferably from about 5 to about 15 wt. %, more preferably about 8 to about 10 wt. % N-acyl-N-methylaminoalkane sulfonate surfactant by weight of the surfactant composition.

14. The process of claim 13, wherein R₁ is H and the waterless base comprises sodium methoxide.

15. The process of claim 12, wherein:
the combining step comprises:
preparing a suspension of the aminoalkane sulfonic acid salt of formula (II) by adding the fatty alkyl ester of formula (III) to the anhydrous alkali salt of an aminoalkane sulfonic acid of formula (II), and
contacting the suspension with the waterless base to form the mixture.

16. The process of claim 12, wherein the combining step comprises combining the waterless base and the fatty alkyl ester of formula (III) to form a premixture and then adding (a) the aminoalkane sulfonic acid of formula (II) or (b) the anhydrous alkali salt of an aminoalkane sulfonic acid of formula (II) to the premixture to form the mixture.

17. The process of claim 12, wherein increasing the temperature of the mixture comprises increasing the temperature of the mixture to from about 65° C. to about 190° C., preferably from about 90° ° C. to about 160° C.

18. The process of claim 12, wherein:
the (a) aminoalkane sulfonic acid of formula (II) is selected from taurine (2-aminoethanesulfonic acid), homotaurine (3-amino-1-propanesulfonic acid) and N-methyl taurine (2-methylaminoethanesulfonic acid);
the (b) the anhydrous alkali salt of an aminoalkane sulfonic acid of formula (II) comprises sodium 2-aminoethanesulfonate, N-methyl taurine sodium salt, sodium 3-aminopropanesulfonate, sodium 3-(N-methylamino)propanesulfonate, and combinations thereof; and
the waterless base comprises a C₁-C₄ alkoxide, preferably sodium methoxide, potassium methoxide, potassium methoxide in methanol solution, or combinations thereof.

19. The process of claim 12, wherein the mixture comprises:
from about 0.90 to about 1.50 moles, preferably from about 0.95 to about 1.20 moles, or more preferably from about 1.00 to about 1.05 moles of the fatty alkyl ester per mole of the alkali salt of an aminoalkane sulfonic acid; and
from about 1.00 to about 1.50 moles, preferably from about 1.02 to about 1.20 moles, and more preferably from about 1.05 to about 1.10 moles of the waterless base per mole of (a) an aminoalkane sulfonic acid of formula (II);
from about 0.01 to about 0.5 moles, preferably from about 0.02 to about 0.2 moles, and more preferably from about 0.05 to about 0.1 moles of the waterless base per mole of (b) an anhydrous alkali salt of an aminoalkane sulfonic acid of formula (II); or
both.

20. The process of claim 12, further comprising adding the N-acyl aminoalkane sulfonate surfactant to water to form a composition comprising greater than 20 wt. %, preferably greater than 25 wt. %, more preferably greater than 30 wt. % of the N-acyl aminoalkane sulfonate surfactant by weight of the composition.
